(19) United States
(12) Patent Application Publication
(10) Pub. No.: US 2014/0276234 A1
(43) Pub. Date: Sep. 18, 2014

(54) INTRAUTERINE MEASUREMENT DEVICE

(71) Applicant: HOLOGIC, INC., Marlborough, MA (US)

(72) Inventors: Keith L. Hines, Dover, MA (US); Robert F. Rioux, Ashland, MA (US); Dominic Hulton, Boston, MA (US); Edward Evantash, Dover, MA (US); Daniel A. Beaudet, Lexington, MA (US); James R. Woodman, Hopkinton, MA (US); Joe Zimo, Arlington, MA (US); Christopher Kaiser, Holliston, MA (US)

(73) Assignee: HOLOGIC, INC., Marlborough, MA (US)

(21) Appl. No.: 13/800,040

(22) Filed: Mar. 13, 2013

(51) Int. Cl. A61B 5/107; A61B 1/303; A61B 5/00

(52) U.S. Cl. CPC A61B 5/1076; A61B 5/4325; A61B 1/303; A61B 5/1079
USPC 600/591

(57) ABSTRACT

Uterine sounds are provided including an insertion member having a distal end, a proximal end, and a lumen, wherein the insertion member is configured for insertion through an endocervical canal. A measurement member is provided having a distal end and a proximal end, the measurement member configured to move within the lumen of the insertion member, where the distal end can protrude from the distal end of the insertion member and is configured for insertion to approximately the fundus of a uterine cavity. An expansion member is also provided transitionable between an unexpanded and expanded state, wherein the unexpanded position is configured to pass an internal cervical os, and the expanded position is configured to prevent passage. Once the expansion member is positioned, the measurement member is positioned relative to the insertion member to establish a measurement of a depth of the uterine cavity.

### BACKGROUND

A variety of intrauterine medical devices can be employed to treat a variety of conditions in patient populations. These medical devices are often inserted through a patient's cervix and then used, for example, within an endometrial cavity to treat the patient.

The human uterine cavity is approximately triangular in shape and relatively flat, much like an envelope. The cavity is entered via the endocervical canal. The proximal end of the canal, the external cervical os, opens to the vagina while the distal end, the internal cervical os, opens to the uterine cavity. The tip of the triangular-shaped uterine cavity is located at the internal cervical os, while the base is defined by the openings that lead to the fallopian tubes, the tubal ostia. Sounding the uterus, i.e., determining a measurement from the fundus of the uterine cavity to the external cervical os has traditionally been a blind procedure. A physician can insert a measuring device or “uterine sound” transcervically and advance the device until it reaches the fundus. The length from the fundus to the external cervical os can be measured directly using graduations stamped on the shaft of the sound. In some conventional approaches, the physician relies upon tactile feedback to determine when the uterine sound has reached the fundus and/or the external cervical os.

Conventional uterine sounds can be constructed to be approximately 3.5 mm in diameter with a working length of roughly 25 cm, and have a flattened handle portion the physician can grasp. The uterine sound can be substantially rigid in the axial direction and somewhat flexible out of plane, transverse to its axis, in order to reach the fundus and provide the physician the tactile sensation of touching the fundus.

Determining the contours of a patient's internal physiology can be important in properly treating and/or employing medical devices within the unique anatomical conditions found in each patient. Some conventional devices have attempted to provide measurements of patient's internal physiology to assist physicians with employing such devices. In particular, conventional gynecological instruments have been developed to define the contours of internal anatomy. Some conventional approaches and devices are overly reliant on a physician's ability to respond to subtle tactile feedback from internal structures to obtain accurate measurements and to capture information on anatomical conditions within patients.

### SUMMARY

Conventional reliance on a physician's ability to detect subtle tactile feedback can result in inconsistent measurements of a patient's internal cavities. It is realized that improving the ability to map, accurately and consistently, internal anatomy can be of benefit in use of devices and/or treatment options that operate within, for example, the uterine cavity. Accordingly, disclosed are uterine sounding devices and methods for measurement that reduce the need for detecting tactile feedback and/or that incorporate unambiguous reference points for improving accuracy of measurement. Thus, uterine sounding devices and methods for measurement are disclosed, which can improve the accuracy and consistency of measurement of internal anatomy.

In one embodiment, a uterine measurement device is provided for obtaining accurate measurements of a dimension of a uterus. The uterine measurement device can include a distal tip for contacting the fundus and an expansion element for establishing a position of an insertion member of the sound. In one embodiment, the expansion member is a balloon that is inflated once positioned in the uterus. By withdrawing the inflated balloon so as to contact the internal cervical os, an operator can accurately position a proximal surface of the balloon against the internal cervical os. The length of the uterus can be determined by ascertaining the relative distance between the distal tip and the proximal surface of the expansion element (proximal relative to the internal side of the internal cervical os).

In some embodiments, the insertion member is constructed and arranged for insertion into and through an endocervical canal passing the internal cervical os into the uterine cavity. Once a distal end of the insertion member is inserted into the uterine cavity and past the internal cervical os, the expansion element can be expanded to a size greater than the opening of the internal cervical os. The insertion member can be drawn out of the endocervical canal until a proximal wall of the expansion element contacts the internal cervical os (proximal determined relative to the internal cervical os). In some implementations, this approach for identifying a position of internal anatomy provides improvement over conventional procedures. A direct measurement of the length of the uterus can then be obtained based on the relative position the expansion member and the distal tip.

The distal tip may be incorporated into or be part of an inner or measurement member that is configured to move within a lumen of the insertion member. The relative position of the measurement member with respect to the insertion member can be shown on a proximal portion of the insertion member. In some examples, the relative position can be shown using graduations on the insertion member, and measurements of uterine length can be obtained by reading an indicator showing relative position on the graduations.

According to another aspect, provided are uterine measurement devices and methods for measurement that reduce the need for tactile feedback through use of internal visualization devices. In some embodiments, an insertion member of a uterine measurement device can be fabricated out of a translucent material. The translucent material is configured to enable, for example, a camera to visualize internal tissue through the translucent material of the insertion member. The insertion member can also be configured with an open channel or hollow chamber, along which, a hysteroscope can be advanced. The hysteroscope can be configured to display internal tissue boundaries, for example, on a monitor or a computer display among other options. A physician can then read graduations on the insertion member that appear proximate to the tissue boundaries, providing measurements of uterine cavity length and/or length of an endocervical canal.

According to one embodiment, a uterine measurement device is provide. The device comprises an insertion member having a distal end, a proximal end, and a lumen, the distal end of the insertion member being configured for insertion into and through an endocervical canal and into an opening of a uterine cavity, an expansion member disposed near the distal end of the insertion member, the expansion member being constructed and arranged to selectively have an expanded state that allows for insertion of the distal end of the insertion member through the endocervical canal and into the opening of the uterine cavity and configured to have an expanded state having a geometry larger than the opening to the uterine cavity, and a measurement member moveable relative to the insertion member, wherein the measurement member is constructed and arranged to selectively extend beyond the distal end of the insertion member, wherein the measurement member is at least as long as the uterine cavity.

According to one embodiment, the expansion member has a diameter of about 0.575 inches in the expanded state. According to one embodiment, the expansion member has a diameter of greater than about 0.454 inches and less than about 0.680 inches in the expanded state. According to one embodiment, the expansion member has a diameter of greater than about 0.533 inches and less than about 0.589 inches in the expanded state. According to one embodiment, the expansion member includes deployable wings. According to one embodiment, the deployable wings in the deployed state are configured to collapse in response to exceeding a threshold seating force. According to one embodiment, the device further comprises a deployment mechanism configured to selectively transition the deployable wings between a contracted state and a deployed state.

According to one embodiment, the deployment mechanism includes at least one of a ring structure configured to deploy the deployable wings in response to rotation, a spring, and a collar configured to accept axial force and redirect the axial force into lateral force upon the deployable wings. According to one embodiment, the device further comprises a tether connected to a distal end of the expansion member, wherein the expansion member is configured to transition between a contracted position and an expanded state responsive to application and release of force directed through the tether.

According to one embodiment, the expansion member is configured to seat near the internal os at a location within about 0.5 cm by applying a target seating force. According to one embodiment, the seating force is about 0.4 to 2.0 lbs. According to one embodiment, the insertion member further comprises graduations marked on at least a portion of a length of the insertion member for indicating relative movement of the measurement member to the insertion member, the relative movement corresponding to the measurement of the depth of the uterine cavity.

According to one embodiment, the device further comprises a control knob located near the proximal end of the insertion member, and a slot formed in a proximal region of the insertion member and adjacent to the graduations and configured to receive the control knob, wherein the control knob is movable within the slot to advance and retract the measurement member within the lumen of the insertion member, wherein a position of the control knob relative to the graduations indicates the dimensions of the uterine cavity with the distal end of the measurement member positioned at approximately the fundus of the uterus.

According to one aspect, a method of using a uterine measuring device, the uterine measuring device including an insertion member and a measurement member, the measurement member slideably disposed within the insertion member, the insertion member including an expansion member is provided. The method comprises advancing the uterine device transcervically until a distal end of the insertion member is within a uterine cavity, expanding the expansion member within the uterine cavity into an expanded state, moving the expansion member until the expansion member contacts an internal cervical os without passing proximally through the internal os, moving the measurement member until the measurement member contacts a fundus, and determining a dimension of the uterine cavity based on a relative position of the insertion member and the measurement member. According to one embodiment, contacting the internal cervical os without passing proximally through the internal os includes applying a seating force of less than about 2.0 lbs.

According to one embodiment, moving the expansion member includes contacting the internal cervical os with a seating force greater than about 0.4 lbs. According to one embodiment, the method further comprises withdrawing the uterine measuring device from the uterine cavity with the expansion member in an expanded state by applying a seating force of at least about 4.0 lbs. According to one embodiment, expanding the expansion member includes an act of actuating a tether connected to the expansion member, and wherein the method further comprises actuating a deployment mechanism configured to transition deployable wings between a contracted position and a deployed position.

According to another aspect, a uterine measurement device is provided. The device comprises an insertion member having a distal end and a set of graduations, wherein the insertion member is configured for insertion into and through an endocervical canal, wherein the insertion member is configured to cooperate with a hysteroscope to capture images of internal tissue boundaries and the graduations disposed on the insertion member proximate to the internal tissue boundaries, wherein the set of graduations is configured to provide a measurement of a length of an imaged internal tissue boundary.

Still other aspects, embodiments, and advantages of these exemplary aspects and embodiments, are discussed in detail below. Embodiments disclosed herein may be combined with other embodiments in any manner consistent with at least one of the principles disclosed herein, and references to “an embodiment,” “some embodiments,” “an alternate embodiment,” “various embodiments,” “one embodiment” or the like are not necessarily mutually exclusive and are intended to indicate that a particular feature, structure, or characteristic described may be included in at least one embodiment. The appearances of such terms herein are not necessarily all referring to the same embodiment.

### BRIEF DESCRIPTION OF THE DRAWINGS

Various aspects of at least one embodiment are discussed below with reference to the accompanying figures, which are not intended to be drawn to scale. The figures are included to provide illustration and a further understanding of the various aspects and embodiments, and are incorporated in and constitute a part of this specification, but are not intended as a definition of the limits of the invention. In the figures, each identical or nearly identical component that is illustrated in various figures is represented by a like numeral. For purposes of clarity, not every component may be labeled in every figure. In the figures:

FIG. 1A shows an isometric view of a uterine measurement device, according to one embodiment;

FIG. 1B shows an isometric view of a uterine measurement device, according to one embodiment;

FIG. 1C shows an isometric view of a uterine measurement device, according to one embodiment;

FIG. 2A shows a side view of a uterine measurement device in a retracted position, according to one embodiment;

FIG. 2B shows a side view of a uterine measurement device in an extended position, according to one embodiment;

FIG. 2C shows a side view of a uterine measurement device in an extended position, according to one embodiment;

FIG. 3 illustrates an example of a uterine measurement device deployed in a uterine cavity, according to one embodiment;

FIG. 4A illustrates an example of an expansion member of a uterine measurement device, according to one embodiment;

FIG. 4B illustrates an example of an expansion member of a uterine measurement device, according to one embodiment; and

FIG. 5A illustrates an example of a uterine measurement device deployed in a uterine cavity, according to one embodiment: and

FIG. 5B illustrates an example of a uterine measurement device deployed in a uterine cavity, according to one embodiment;

FIG. 5C illustrates an example of a uterine measurement device deployed in a uterine cavity, according to one embodiment;

FIG. 6 is a flowchart showing an example process for measuring dimensions of a uterine cavity, according to one embodiment;

FIG. 7 shows a portion of a member of a uterine measurement device, according to one embodiment;

FIG. 8 shows an implementation of a collection cup coupled to a tip of a member of a uterine measurement device, according to one embodiment;

FIG. 9 shows an isometric view of a uterine measurement device including a lockable control knob, according to one embodiment;

FIG. 10 shows an isometric view of a uterine measurement device including locking grooves, according to one embodiment;

FIG. 11A shows side and end views of a full radius tip of a uterine measurement device, according to one embodiment;

FIG. 11B shows side and end views of a chamfered tip of a uterine measurement device, according to one embodiment;

FIG. 11C shows side and end views of a concave tip of a uterine measurement device, according to one embodiment;

FIG. 12A shows a full radius tip of a uterine measurement device producing an axial load on the uterine wall, according to one embodiment;

FIG. 12B shows a chamfered tip of a uterine measurement device producing an axial load on the uterine wall, according to one embodiment;

FIG. 12C shows a concave tip of a uterine measurement device producing an axial load on the uterine wall, according to one embodiment;

FIG. 13A is a top view of an inner or measurement member of a uterine measurement device in a closed position, according to one embodiment;

FIG. 13B is a top view of the inner or measurement member of FIG. 10A in an open position, according to one embodiment;

FIG. 14 is a top view of the end cap of the inner or measurement member of FIG. 10A, according to one embodiment;

FIG. 15 is a top view of the end cap of the measurement member of FIG. 10B, according to one embodiment;

FIG. 16 is a cutaway view of a handle of the measurement member of FIGS. 10A and 10B, according to one embodiment;

FIG. 17 illustrates and example of a uterine measurement device, according to one embodiment;

FIG. 18 illustrates and example of a uterine measurement device, according to one embodiment;

FIG. 19 illustrates and example of a uterine measurement device, according to one embodiment; and

FIG. 20 illustrates a process for measuring internal tissue boundaries, according to one embodiment.

### DETAILED DESCRIPTION

Aspects and embodiments of this disclosure are directed to methods and devices for obtaining measurements of uterine cavity length and a “uterine sound length,” where “uterine sound length” refers to the length from the fundus of the uterine cavity to the external cervical os. Various embodiments provide for direct measurement of the dimensions of the uterus, including, for example, uterine sound length, among other options. In one example, the measurement device can measure a uterine cavity length measured from the fundus of the uterine cavity to the internal side of the internal cervical os, in addition to measuring the uterine sound length. According to some embodiments, an expansion member is provided on the measurement device that can be positioned against the internal side of the cervical os of the patient to provide an unambiguous fixed positional reference. The positional reference can be used to facilitate direct measurements of, for example, the length of the uterine cavity using other elements of the device to determine a distance from the positional reference to another internal landmark. Alternative embodiments of methods and devices for uterine measurement can incorporate direct visualization, rather than tactile feedback, for measuring uterine length and “sounding” of a uterus of a patient.

According to various embodiments, the uterine measurement device includes an insertion member having a distal end, a proximal end, and a lumen. The distal end of the insertion member can include or be connected to an expansion member for positioning the device. In one embodiment, a measurement member having a distal end and a proximal end is disposed within the insertion member and is configured to move within the lumen of the insertion member. In some embodiments, the measurement member can be disposed outside of the insertion member, and the measurement member can translate along the outside of the insertion member. The distal end of the measurement member can selectively protrude beyond the distal end of the insertion member.

According to some embodiments, the uterine measurement device is inserted into and through the endocervical canal with the measurement member fully retracted and the expansion member in its contracted state until the expansion member is positioned within the uterine cavity. The expansion element can then be expanded to a size greater than an opening of the internal cervical os. The uterine measurement device is then withdrawn proximally until the expansion member engages an interior surface of the uterus on the posterior side of the internal cervical os. After the expansion member is seated against the uterine cavity side of the internal os, the insertion member remains in this position for the remainder of the measurement procedure. The measurement member is then extended distally from the distal end of the insertion member until the distal tip of the measurement member contacts the fundus. The user can then obtain direct measurements of uterine cavity length and sound as described in greater detail below.

FIG. 1A shows an implementation of a uterine measurement device 100. The uterine measurement device 100 includes an insertion member 102, a measurement member 104 disposed within a lumen 103 of the insertion member 102, and a control knob 106 coupled to the measurement member 104 for translating the measurement member 104 within the insertion member 102. The insertion member 102 includes a proximal end 125 and distal end 127.

The insertion member 102 includes an expansion member 108 near the distal end 127. In other embodiments, the expansion member 108 can be located at the distal end of the insertion member. The expansion member 108 can be constructed to have an unexpanded state 108A, shown in FIG. 1A, and a contracted state 108B, shown in FIG. 1B. Unexpanded state 108A can include an outer diameter less than, approximately flush with, or minimally bigger than an outer diameter of the insertion member 102. The expanded state can have an outer diameter substantially greater than the outer diameter of the insertion member 102. Further, the outer diameter of the expansion member 108 in the expanded state can be greater than a size of the opening of an internal cervical os. In some embodiments, the expansion member can include a balloon. The balloon can be responsive to an inflation control (not shown). An operator of the device can actuate an inflation control to inflate the balloon into an expanded state (FIG. 1B, 108B).

In some embodiments, an operator can facilitate the identification of the internal cervical os position using the expansion member 108 in conjunction with a dilation process. One or more dilators can be used to achieve a desired cervical opening. Rather than rely on the elastic differences between the internal cervical os and the cervical canal, as in some conventional approaches, the change in diameter provided by the expanded state of the expansion member can be used to identify the internal cervical os. For example, a fixed positional reference can be established where the enlarged diameter of the expansion member can no longer pass through the opening to the cervical canal, or where the enlarged diameter of the expansion member provides resistance to further proximal movement of the insertion member.

In one example, a uterine measurement device can be used to establish a fixed positional reference for determining a dimension of uterine cavity. If the insertion member 102 has a 4 mm diameter, the operator can dilate the patient's cervix to 4 mm and insert the measurement device. As the operator/physician advances the uterine measurement device 100 into the cervical canal, the expansion member 108 in an unexpanded state should pass with minimal resistance through the internal cervical os. In some examples, the physician can rely on her or his experience in determining an approximate insertion distance that places the expansion member 108 through the cervical canal and into the uterine cavity. In other examples, the physician can advance the insertion member with the measurement member fully retracted until the distal tip 116 contacts the fundus. In some further examples, the physician can rely on tactile feedback upon encountering and passing through the cervical canal to determine that the expansion member is within the uterine cavity.

Once the expansion member has passed through the cervical canal into the uterine cavity, it can be expanded. Upon expansion in the uterine cavity, the expansion member can be configured to achieve a diameter of, for example, 5-6 mm or a greater diameter. With the expansion member 108 in an expanded state, the insertion member 102 and the expansion member are now configured to not pass through the internal cervical os back into the cervical canal. In particular, as the device is retracted back toward the cervical canal, the expansion member cannot be drawn beyond the internal cervical os, definitively establishing the position of the device and the internal cervical os.

In further embodiments, minimal dilation can be used in conjunction with smaller diameter devices. Because the expansion member is inserted past the internal cervical os, the insertion diameter of the device can be as little as 2-4 mm, and an expansion diameter of the expansion member can be 1-2 or more mm greater than the insertion diameter to detect the internal cervical os on return passage. In other embodiments, different diameters can be used for the expansion member (including larger diameters), dilation, and the insertion member.

The uterine measurement device 100 can include a handle 118 attached to the proximal end of the insertion member 102. In one implementation, the handle 118 is an extension of the insertion member 102. In another implementation, the handle 118 is a separate component coupled to the insertion member 102. The handle 118 can be configured for operator manipulation including finger grips or other tactile features allowing the user to hold the uterine measurement device 100. In some embodiments, the handle can include an expansion member actuator (not shown). For example, an actuator can be provided for inflating and/or deflating a balloon. In other examples, the expansion member can include wings that protrude from the insertion member upon activation.

In the implementation shown, the insertion member 102 includes a first set of graduations 112 positioned near the proximal end. The first set of graduations 112 can be configured to provide a length measurement of a uterine cavity. The insertion member 102 can optionally include a second set of graduations 114 positioned near the distal end. The second set of graduations 114 are configured to provide a length measurement of an endocervical canal. The unit graduations on each set of graduations 112 and 114 can demarcate unit measurements, for example, in centimeters, millimeters, or some other unit.

The insertion member 102 includes a slot 110 extending longitudinally in the proximal region. The slot can extend radially through one side of a wall of the insertion member 102 from an outer radius to an inner radius, or can extend through both walls of the insertion member 102, e.g., along a diameter of the insertion member 102. The slot 110 houses a control knob 106 coupled to the inner or measurement member 104, such that by moving the control knob 106 along the length of the slot, the measurement member 104 is guided within the lumen 103 of the insertion member 102 and can move between a retracted and an extended position. In one implementation, the slot 110 extends substantially the length of the first set of graduations 112. According to some embodiment, a ball plunger can be integrated in the measurement device to provide finer control of the movement of the measurement member. In one example, the ball plunger can be depressed to guide the measurement member into a patient with more precise force control than use of the control knob alone.

In some embodiments, the control knob 106 can also be constructed to expand the expansion member 108. For example, in one embodiment the control knob 106 can be translated within slot 110 to transition the expansion member 108 from contracted state 108A to expanded state 108B. In particular, pulling the control knob 106 towards the handle 118 results in an expansion force being applied at the distal end of the insertion member, which can be delivered to the expansion member. For example, with reference to FIG. 1C, in response to translating the control knob 106 proximally, wings 121A, 121B may be deployed. Shown in FIG. 1C is an embodiment having two deployable wings. Other embodiments can include additional wings. In one example, a measurement member can include 3, 4, or more wings, for example, to increase tissue contact area. According to one embodiment, additional wings can also be employed to decrease a deployment force relative to embodiments having fewer wings.

In another example, lumen 103 can include a slot extending circumferentially and the control knob 106 can be directed to a circumferential slot to lock the wings in a deployed position. For such embodiments, the control knob 106 can also be turned to align the control knob 106 for travel within the slot 110. Once a measurement is obtained, the control knob can be rotated to return the expansion member, such as the wings to a non-deployed position, facilitating removal of the device. According to one embodiment, wings 121A and 121B can be constructed with a bulbous or diamond shape. According to some embodiments, the diamond shape provides more rigid tactile feedback upon contact with internal os, as compared to the soft, gradual feedback provided by the bulbous shape.

According to one aspect, various implementations of a measurement device are configured to optimize the dimensions of the wings to match against patient anatomy across a large patient population. In one example, the diameter of the wings when deployed (measured from crease in 121A to crease in 121B) and a respective angle are constructed and arranged to fit comfortably for the large patient population. According to one embodiment, the wing diameter is 0.575 inches and the angle provided is 60 degrees (+/−10 degrees) measured from either side of the bend. According to some implementations, the diameter of 0.575 inches and the angle of 60 degrees (+/−10 degrees) provides benefit in terms of accuracy and repeatability of measurements with less user variability.

Various embodiments can include safety features associated with the deployable wings. For example, in the event the expansion member will not contract, the wings are constructed and arranged to collapse towards the distal end. According to one embodiment, when the device is withdrawn proximally, and the force applied to the device exceeds a maximum seating force (e.g., 3.0 lbs) the wings collapse towards the distal end of the device folding up similarly to the canopy of an umbrella inverting in response to excessive wind. Under normal conditions, a typical seating force can vary between 0.4 and 2.0 lbs, thus the wings can be constructed with different maximal seating force thresholds.

In some embodiments, the wings are constructed to form a diamond shape. The bends of the respective wings can be rounded to prevent injury, as the bends scrape tissue just before seating. In one example, the wings are constructed of a thermoplastic polyester elastomer that provides comfort, minimizes injury potential, and can be constructed with the maximal seating force threshold.

According to some implementations, the uterine measurement device, and in particular the expansion member, can be constructed to have the following properties summarized in TABLE 1. TABLE 1 summarizes the results of bench tests conducted to show seating position of the expansion member as a function of expansion member diameter and an expected seating force. The tests were performed at each end of a range of expected seating forces. In each case, the expansion member was made of a thermoplastic polyester elastomer. The seating position is reported relative to true zero (i.e., the optimum seating location of the expansion member so as to obtain a measurement of the actual length of the uterine cavity). In particular, the first column provides the diameter of the expansion member of the uterine measurement device. The second column provides the seating position of the expansion member with respect to true zero when positioned according to lowest expected seating force. The third column provides information on the positioning of the expansion member with respect to true zero when positioned according to the highest expected seating force.

TABLE 1

| | Location relative to | Location relative to |
|---|---|---|
| | true zero at | true zero at |
| Diameter of | approximately .4 | approximately 2.0 |
| Expansion Member | lbs of seating force | lbs of seating force |
| .360″ | +.071″ (+.2 CM) | N/A pulled through |
| | | at .99 lbs |
| .454″ | +.005″ (+.0 CM) | +.316″ (+.8 CM) |
| .575″ | −.056″ (+.1 CM) | +.170″ (+.4 CM) |
| .680″ | −.314″ (−.8 CM) | +.368″ (+.9 CM) |
| .825″ | −.505″ (−1.3 CM) | +.331″ (+.8 CM) |

Thus, according to TABLE 1, one embodiment of the uterine measurement device can be constructed and arranged to include an expansion member having a diameter preferably in a range of greater than 0.454″ and less than 0.680″ to identify (within +/−0.9 cm) the true length of the uterine cavity over a desired operating range of a seating force and more preferably in a range of greater than 0.553″ and less than 0.589″ (accurate within about +/−0.5 cm) and more preferably a diameter of about 0.575″.

In the uterine measurement device 100 shown in FIG. 1A, the insertion member 102 is configured for insertion into and through the endocervical canal. In some embodiments, the uterine measurement device 100 can be disposable. The insertion member 102 can be formed from injection molded thermoplastic, metal, or other material. In one implementation, the insertion member 102 can be formed from plastics such as ABS, polystyrene, Peek, polycarbonate, or Ultem. In another implementation, the insertion member 102 can be formed by injection molding two longitudinal halves, which are then attached together, for example, through the use of an adhesive or other bonding technique. Alternatively, the insertion member 102 can be machined from a solid rod or tube of material. The insertion member 102 can be substantially rigid in a compressive direction axially with respect to the distal and proximal ends as well as non-axially. According to one embodiment, the insertion member 102 is constructed of a thermoplastic polyester elastomer. Various implementations provide different materials that are flexible but rigid and include shape memory.

As discussed, the measurement member 104 is movable and has a proximal and a distal end and is configured to move within the lumen 103 provided by the insertion member 102. The measurement member 104 includes a tip 116 at the distal end. The tip 116 can be configured to be atraumatic to reduce a risk of injury when contacting uterine tissue (e.g., to reduce a risk of perforating the uterine wall). For example, as shown in FIG. 1A, the tip 116 can include a rounded surface that distributes the pressure generated by contact between the tip 116 and uterine tissue over a larger surface area, reducing the risk of damage. The measurement member 104 can also be flexible to allow a degree of bending necessary to locate the fundus of a curved uterus.

According to one embodiment, the control knob 106 is configured to allow the operator to control movement of the measurement member 104 relative to the insertion member 102. In one implementation, the control knob is fixedly attached to the measurement member 104, such that a movement of the control knob 106 provides a corresponding movement of the measurement member 104. For example, if the control knob 106 is moved (e.g., through operator manipulation) toward the distal end of the insertion member 102, the measurement member 104 extends from the distal end of the insertion member 102. The control knob 106 can be configured to move along the outside of the insertion member 102. For example, the control knob 106 can include a ring shape surrounding the insertion member 102 with a connector (e.g., pin connector) to the measurement member 104 extending through the slot 110. The slot 110 can thereby function as a guide, defining the range over which the control knob 106 and the measurement member 104 can move. The control knob 106 can be configured to facilitate operator manipulation, for example, including finger grips or other tactile features allowing the user to control the movement of the control knob.

In one implementation, the control knob 106 can lock the measurement member 104 of uterine measurement device 100 in a retracted position. For example, a notch can be included orthogonal to the end point of the slot 110 at the proximal end of the uterine measurement device 100 such that a rotation of the control knob 106 in a direction of the notch can lock the measurement member 104 and a reverse rotation from the locked position can unlock the measurement member 104. In further embodiments, additional slots can be constructed on the insertion member 102. In one example, an additional slot is provided to permit the control knob and inner member to move toward the proximal end of the insertion member. The force applied to the control knob towards the proximal end of the insertion member can be transmitted to the distal end of the insertion member and/or the expansion member. For example, the force can be transmitted to the distal end to actuate wings into an expanded state (FIG. 1C, 108C).

In another implementation, the measurement member 104 can be threaded within the insertion member 102, and a control knob can be rotated by a user to thread the measurement member 104 between an extended and retracted position. In one embodiment, the control knob includes an inner thread that mates with a thread formed on the exterior of the measurement member 104, and rotating the control knob translates the measurement member 104 axially. It is understood that other configurations can be used to translate the measurement member 104 relative to the insertion member 102 and to deploy an expansion member, as the techniques described herein are merely exemplary.

FIGS. 2A and 2B illustrate the uterine measurement device 100 in the retracted and extended positions of the measurement member 104, respectively. In FIG. 2A, the uterine measurement device 100 is shown with the measurement member 104 in the retracted position. In the retracted position, the control knob 106 is positioned toward the proximal end of the slot 110 in the insertion member 102. The measurement member 104 is contained within the lumen provided by the insertion member 102 such that only the tip 116 of the measurement member 104 protrudes from the proximal end of the insertion member 102.

In FIG. 2B, the uterine measurement device 100 is shown in the extended position of the measurement member 104. In the extended position, the control knob 106 is moved from the proximal end of the slot 110 toward the distal end of the slot 110 formed in the insertion member 102. Consequently, as shown in FIG. 2B, the measurement member 104 is shown extended from the insertion member 102. In one implementation, a position of the control knob 106 relative to the first set of graduations 112 provides a measurement of the extended distance of the measurement member 104, which, in use, can correlate to the length of the uterine cavity.

In one implementation, the uterine measurement device 100 can be disposable. As discussed, the insertion member 102 can be formed from injection molded thermoplastic, metal, or other material. The insertion member can be molded to include channels or tracks in which a control knob 106 can moveably operate to control extension and retraction of a measurement member 104.

The measurement member 104 can also be composed of injection molded thermoplastic. The plastic material can include polystyrene, LDPE, HDPE, a blend of LDPE/HDPE, polycarbonate, ABS, Peek, Delrin, or other suitable materials. The tip 116 and the shaft of the measurement member 104 can be assembled from separate components or molded as a single component. The measurement member 104 can be formed to include a curvature suitable for easing passage of the measurement member 104 through the uterus. The curvature of the measurement member 104 can be configured in any number of shapes and degrees of curvature, including, for example, an average curvature of a uterus.

FIG. 2C illustrates the uterine measurement device 100 in the extended position of the inner or measurement member 104, with the expansion member 108 shown in an expanded state 108D. The expansion member can be constructed of a balloon that expands into an expansion position. In other embodiments, the expansion member can include multiple wings configured to transition between an unexpanded state and an expanded state having a diameter greater than the internal cervical os.

The uterine measurement device 100 can also include, for example, a plunger 120 within the handle 118. In one example, upon depression of the plunger 120, air or other fluid can be forced into the balloon. The forced air can transition the balloon into an expanded state. In some embodiments, lumen 103 can include a channel (not shown) connecting the expansion member and the plunger 120. The plunger can be operatively connected to a shaft or piston. By depressing the plunger, the shaft or piston can travel into the channel increasing pressure within the channel and resulting in expansion of the balloon into an expanded state. In some examples, the channel can be filled with air or liquid. The movement of the piston or shaft can force air or liquid into the balloon resulting in the expanded state. The plunger can include locking mechanisms for keeping the plunger in a depressed state until released. For example, a key lock mechanism can be built into the handle 118. Further, the plunger can include twist lock structures for locking the plunger in position once depressed. Once the locking mechanism is released and the plunger returns to a non depressed state, elastic properties of the balloon can be configured to return the balloon to an unexpanded state.

In another example, FIG. 3 illustrates a uterine measurement 300 device deployed in a uterine cavity 301. The uterine measurement device includes an insertion member 302 and lumen 303 configured to pass through the endocervical canal 320 defined between the external cervical os 322 and the internal cervical os 324. The insertion member 302 can include or be connected to an expansion member 308, configured to pass through the endocervical canal 320 and beyond the internal cervical os 324 in an unexpanded state (not shown) into the uterine cavity. Upon passage into the uterine cavity, an operator can actuate the expansion member 308 into an expanded state 308A. Various actuation devices can be used to transition the expansion member 308 between an unexpanded and expanded state, including for example, a plunger fluidly connected to the expansion member, a tether configured to apply a force to the expansion member in a direction out of the endocervical canal, a screw drive, a twist lock structure, etc. Shown by way of example, FIGS. 4A-B illustrate another implementation of a uterine measurement device in respective expansion and non-expansion positions. In FIGS. 4A-B, a tether (e.g., at 407 shown in dashed line) is connected to the expansion member 408, which actuates the expansion member between the unexpanded state 408A and an expanded state 408B, of FIG. 4B. Referring to FIG. 3, the uterine measurement device can include a measurement member 304. The measurement member can be configured to extend out of the insertion member to a position approximately at the fundus 326 of the uterine cavity 301.

As discussed, FIGS. 4A-B illustrate examples of expansion members of uterine measurement devices. For example, uterine measurement device 400 can include an insertion member 402 configured to pass through an endocervical canal, and into a uterine cavity. Device 400 can include a measurement member 404, similar to the measurement member discussed with respect to FIG. 1 (e.g., 104). Measurement member 404 can operate within a lumen 403 defined by the insertion member 402. In some embodiments of the uterine measurement device, the measurement member 404 can include an expansion member 408 constructed of an elastically deformable material. The expansion member can be a flexible tube at 408 configured to travel within the insertion member 402 and deploy with the measurement member 404 into the uterine cavity. In some embodiments, the expansion member can be configured to travel within the lumen of the insertion member as the expansion member travels from a non-extended position to an extended position. Further, the expansion member can be attached to a tether (not shown), configured to transition the expansion member 408 into an expanded state upon an application of force to the tether, and transition the expansion member 408 into an unexpanded state 408A upon release of the tether. In some embodiments, the expansion member 408 is resilient or elastic, such that release of the tether causes the expansion member 408 to return to the unexpanded state 408A.

In some embodiments, the expansion member may be a flexible tube which can include slits cut into a distal portion to bias expansion of the flexible tube in response to forces applied by, for example, a tether. In one embodiment, upon application of force exerted through pulling the tether connected to the distal end of the flexible tube, the flexible tube can be configured to expand. In one example, the flexible tube can be expanded to a diameter of 1-2 mm greater than the diameter of the insertion member 408. More particularly, the expanded diameter is configured to have a size configured to not pass the internal cervical os back into the endocervical canal. In some examples, the expanded diameter can be greater than 1-2 mm over the diameter of the insertion member. For example, FIG. 4B illustrates the uterine measurement device 400 with the expansion member 408 (e.g., a flexible tube) having a portion in an expanded state 408B.

FIGS. 5A-B illustrate another embodiment of a uterine measurement device, having additional configurations of an expansion member. In FIG. 5A, there is illustrated an embodiment of a uterine measurement device 500 having an expansion member 508 comprising deployable wings 521A-521B. The measurement device 500 includes an insertion member 502, lumen 503, inner or measurement member 504, and expansion member 508 shown in an expanded state 508A. The inner member 504 can be connected to a control knob 506 that when moved towards a distal end 509 of the insertion member 502, the inner member 504 beyond the distal end of the insertion member 502.

The device can include a first set of graduations 512 positioned near the proximal end 511 of the insertion member 502. The first set of graduations 112 can provide a set of unit graduations configured to provide a length measurement of a uterine cavity. The insertion member 102 can optionally include a second set of graduations 114 positioned near the distal end 509. The second set of graduations 114 can provide a set of unit graduations configured to provide a length measurement of an endocervical canal. For example, the second set of unit graduations can be used as discussed below to determine a length of an endocervical canal. The unit graduations on each set of graduations 512 and 514 can demarcate unit measurements, for example, in centimeters, millimeters, or some other unit.

Like the measurement devices describe above (e.g., 100 and 300), device 500 is configured to pass through an endocervical canal with the expansion member in an unexpanded or retracted position. FIG. 5B illustrates the device 500 partially within a uterine cavity 501. In FIG. 5B, a distal tip 516 of the device is in contact with a fundus 526 of the uterine cavity. The expansion member is shown in an unexpanded state 508B. In various embodiments, an operator can actuate the expansion member 508 to achieve an expanded state (e.g., 508C, FIG. 5C). As shown in FIG. 5B, the device 500 has been inserted transcervically into and through endocervical canal 520 defined between the internal cervical os 524 and the external cervical os 522. Once the operator has inserted the device 500 to the fundus 526, the operator may actuate the expansion member 508 to achieve an expanded state (e.g., as shown in FIG. 5C, 508C). In another example, once the operator has inserted the device 500 to a position within the uterus, the operator may actuate the expansion member 508 to achieve the expanded state (e.g., 508C).

According to one embodiment, the device 500 can be configured to permit an operator to withdraw the insertion member transcervically back toward the endocervical canal while maintaining the distal tip 516 at the fundus 526. For example, the operation can hold the control knob (e.g., 506, FIG. 5A) in position while withdrawing the insertion member 502. The expansion member 508 in the expanded state 508C is configured not to pass the internal cervical os without substantial resistance. Upon reaching contact with the internal cervical os (e.g., portions of wings 521A-B), the operator can definitively establish the cervical canal length. With the distal tip 516 still positioned at approximately the fundus 526, the operator can also read a measurement of the length of the uterine cavity from the graduations (512) on the lumen 503 of the device, for example, at the control knob 506. Once the operator has a measurement of the dimensions of the uterine cavity, the operator can transition the expansion member 508 into the unexpanded state for removal of the device 500. It is to be appreciated that the inner member 504 may be retracted prior to removing the device but need not be.

FIG. 6 illustrates one exemplary process 600 for using any of the herein disclosed uterine measurement devices to measure the uterine cavity dimensions. For illustrative purposes, the process 300 shall be described in reference to the implementation of the uterine measurement device shown in FIGS. 1-2, however, it shall be understood that the process 600 can be carried out using any of the uterine measurement devices (including without limitation, 300, 400, 500, among other examples).

An operator, such as a physician or other medical professional, transcervically inserts the uterine measurement device (step 602). The uterine measurement device can be inserted in the retracted position with the measurement member 104 within the lumen of the insertion member 102. Further, during insertion at 602, the expansion member is maintained in an unexpanded state. In one implementation, the operator can first dilate the cervix to a diameter less than or equal to the diameter of the insertion member 102 of the outer sheath. As has been noted herein for various embodiments of the measurement device, the expansion member is configured to achieve a diameter greater than the dilation diameter in an associated expansion position.

The operator advances the uterine measurement device through the endocervical canal until the distal end 116 of insertion member 102 and the expansion member 108 pass the internal cervical os and are positioned within the uterine cavity (step 604). In some examples, the device can be inserted anywhere in the uterine cavity up to and until the distal end 116 reaches to approximately the fundus of the uterine cavity.

After inserting the insertion member 102 of the uterine measurement device 100 into the uterine cavity, the operator actuates the expansion member 108 (step 606). Expansion member 108 obtains an expansion position (e.g., 108B, FIG. 1B) responsive to actuation by the operator. Once the expansion member is positioned in a respective expansion position, the operator withdraws the device 100 in a direction away from the patient's uterine cavity toward the endocervical canal. As discussed, the device is configured to only travel until the expansion member meets the internal side of the patient's internal cervical os. Based on the diameter of the expansion member, the expansion member can be configured not to pass the internal cervical os, thereby positioning the expansion member at the internal cervical os (step 608). Alternatively, for other embodiments of the expansion member with a smaller expanded geometry, at least substantial resistance can be detected upon contact between the expansion member and the internal cervical os.

Once the expansion member is in position at the internal cervical os, the operator can optionally extend the tip 116 of the measurement member 104 of the uterine measurement device to approximately contact the fundus of the uterine cavity (step 610). In some embodiments, rather than position the expansion member and then extend the tip 116 (e.g., as in optional step 610), the operator can first position the tip of the measurement member to be substantially at the fundus of the uterine cavity and then withdraw only the insertion member 102 of the device in step 608. For example, the operator can advance the device at 604 until the tip 116 reaches the fundus and expand the expansion member (e.g., 606). The operator can then retract only the insertion member 102 until the expansion member comes in contact with the internal cervical os. For example, the operator could hold the control knob 106 in place while retracting the insertion member. Once the expansion member mates with the internal side of the internal cervical os, the operator can obtain a dimension of the uterine cavity by reading the distance indicated by the hash marks on the insertion member (Step 612).

It is to be appreciated that during optional step 610, the operator can extend the measurement member 104 by manually moving the control knob 106 coupled to the measurement member 104, through the insertion member 102. For example, the operator can advance the control knob 106 along the length of the insertion member 102 toward the distal end in order to extend the inner member beyond the distal end of the insertion member 102 by a corresponding amount. The operator locates the fundus of the uterine cavity by tactile feel of axial resistance from the measurement member 104 once the tip 116 of the inner member contacts the uterine wall at the fundus.

Once tip 116 is positioned at the fundus, the operator can directly measure the length of the uterine cavity (step 612). The length measurement can be determined from relative positions of the insertion member and measurement member. The position of the control knob 106 in the slot 110 indicates the measurement of the length of the uterine cavity. As discussed above, in the implementation shown in FIGS. 1-2C, the insertion member 102 includes a first set of graduations 112 that indicate different measurement amounts. The position of the control knob 106 relative to the graduations can provides a direct measurement of length for the uterine cavity.

Optionally, the operator can directly measure the length of the endocervical canal (step 614). The operator can measure the length of the endocervical canal according to a second set of graduations 114 positioned near the distal end of the insertion member 102. The length of the endocervical canal is measured from the portion of the expansion element proximal to the internal side of the internal cervical os to the external os. In one implementation, the device can include a collar slideably disposed on the insertion member 102. In one example, the operator can move the collar along the insertion member 102 until the external os is reached. The position of the collar relative to the second set of graduations 114 provides an indication of the length of the endocervical canal.

After measuring the uterine cavity length (e.g., 612), and prior to extraction of the device 100, the operator can actuate the expansion member 108 to transition the expansion member to an unexpanded or retracted position (step 616). Further, the operator can optionally retract the measurement member 104 back within the insertion member 102 for extraction of the uterine measurement device 100 from the patient (step 616). Alternatively, the operator can leave the inner member in the extended position, for example, to read the measurement at a later time. In one implementation, the control knob 106 can be locked into position, with the inner member extended, such that the measurement position is maintained for later review. The uterine measurement device 100 can then be withdrawn transcervically (step 620).

FIG. 7 shows a detailed view of a distal portion of one implementation of an inner or measurement member 700. The inner member 700 includes a shaft 702 (partially shown) and a tip 704. The shaft 702 has a ribbon shape having a rectangular cross section with a width 706 and a height 708. The rectangular cross section of the shaft 702 provides a preferential bending plane for the measurement member 700. The width 706 is greater than the height 708, such that the measurement member 700 has a greater flexibility along a plane including the width 706 than along a plane including the height 708. The measurement member 700 can be configured to provide the preferential bending along the plane of the triangular uterine cavity, which can be curved upwards or downwards out of the plane. The flexible measurement member therefore can flex in order to accurately locate the fundus of the uterine cavity when the uterus is curved upwards or downwards. Additionally, the lesser flexibility provided in the plane including the height 708 reduces the chance of bending the measurement member 700 such that the tip 704 enters either of the fallopian tubes.

In an alternative implementation, the measurement member can be formed from one or a combination of materials in order to provide variable flexibility along the length of the measurement member. The variable flexibility of the measurement member can be configured to provide a greater degree of flexibility along the distal end of the measurement member and a lesser degree of flexibility at the proximal end. In one implementation, the degree of flexibility of the measurement member can incrementally increase from the proximal end to the distal end. In one implementation, the variable flexibility can be provided geometrically. For example, the shaft of the measurement member can taper from the proximal end to the distal end in order to provide greater flexibility at the distal end.

FIG. 8 shows one implementation of a tip 802 of an inner or measurement member 800. The tip 802 is attached to the distal end of a shaft 804 (partially shown) of the measurement member 800. The tip 802 is configured in a cup shape having a convex shaped outer surface 806 and a concave inner surface 808. An edge 810 demarcates the rim of the cup separating the outer surface 806 and the inner surface 808. The convex outer surface 806 is configured to provide an atraumatic surface for contacting the uterine wall. The concave inner surface 808 is configured to collect endometrial tissue from the uterine wall as the edge 810 scrapes along the uterine cavity when the measurement member 800 is retracted. The concave inner surface 808 collects the tissue scrapings for testing or other purposes by an operator or other individual such as a lab technician.

FIG. 9 shows another implementation of a uterine measurement device 900. The uterine measurement device 900 is similar to the uterine measurement device 100 shown in FIG. 1, and also includes an insertion member 902, inner or measurement member 904, and a control knob 906. The insertion member 902 includes a lumen 903 having a proximal and distal end and an expansion member 908 at the distal end of the insertion member 902.

The uterine measurement device 900 also includes a handle 918 attached to the proximal end of the insertion member 902. The handle 918 can include a plunger 920 to actuate the expansion member 908 between a first unexpanded state and a second expanded state. Upon depression, plunger 920 can be configured to lock in place, locking the expansion member in a respective position. In one embodiment, an operator can re-depress the plunger, releasing the lock and allowing the plunger 920 to return to an un-depressed position. The release of the plunger can be configured to actuate the expansion member from, for example, an expanded state to an unexpanded state.

The insertion member 902 can also include a first set of graduations 911 along the proximal end. The first set of graduations 911 can provide a set of unit graduations configured to define a length measurement of a uterine cavity. The insertion member 902 can optionally include a second set of graduations 913 along the distal end. The second set of graduations 913 can provide a set of unit graduations configured to provide a length measurement of an endocervical canal.

The insertion member 902 also includes a slot 910 along the proximal end. The slot can extend radially through a single wall of the lumen formed by the insertion member 903 from the outer diameter to the inner diameter, or through both walls of the lumen 903, e.g., along a diameter of the lumen. The slot 910 allows the control knob 906 to attach to the measurement member 904.

The uterine measurement device 900 includes at least the following feature that is not included in the device 100 shown in FIG. 1. A movable element is coupled to the insertion member 902 for measuring the length of the endocervical canal according to the second set of graduations 913. In the implementation shown, the element is a collar 912. However, other configurations of the movable element are possible. During a measurement operation, the operator can manually move the collar 912 along the insertion member 902 toward the distal end until the external os of the cervix is reached. In one implementation, the collar 912 is configured as a ring that can slide along the outer surface of the insertion member 902. After removing the uterine measurement device 900 from the patient, the operator can view a direct measurement of the endocervical canal length according to the position of the collar 912 relative to the second set of graduations 913.

The uterine measurement device 900 also can include the following feature. The control knob 906 can be lockable, allowing the operator to control movement of the measurement member 904 relative to the insertion member 902. In one implementation, the control knob is fixedly attached to the measurement member such that a movement of the control knob 906 provides a corresponding movement of the measurement member 904. For example, if the control knob 906 is moved (e.g., through operator manipulation) toward the distal end of the insertion member 902, the measurement member 104 extends from the distal end of the insertion member 902. The control knob 906 can move along the outside of the insertion member 902. For example, the control knob 906 can include a ring shape surrounding the insertion member 902. The control knob 906 can be attached to the measurement member 904 through the slot 910 using, for example, a pin connector.

Additionally, the lockable control knob 906 includes a locking collar 914 configured to lock the control knob 906 in place along the insertion member 902. The locking collar 914 allows the operator to lock the control knob at any position within the movable range of the control knob 906 along the insertion member 902. For example, the operator can lock the control knob 906 once the fundus has been located such that the uterine measurement device 900 can be withdrawn and the uterine dimensions recorded later according to the locked position of the control knob 906. In one implementation, the locking collar 914 is configured to tighten around the insertion member 902 to lock the control knob 906. For example, the locking collar 914 can be a rotatable collar positioned at the proximal end of the control knob 902. Rotation of the locking collar 914 tightens the locking collar 914 around the insertion member 902 providing a friction hold of the control knob 902. Rotation of the locking collar 914 in the opposite direction can then untighten the locking collar 914, releasing the control knob 902. Other locking mechanisms can be used, for example, a pin vise clamp, threaded collar or other structure.

FIG. 10 shows another implementation of a uterine measurement device 1000. The uterine measurement device 1000 includes an insertion member 1002, inner or measurement member 1004, tip 1016, handle 1018, expansion member 1008, and control knob 1006. The insertion member 1002 includes a slot 1010 and a series of locking grooves 1020. The slot 1010 runs along a portion of the axis of the insertion member 1002 and provides for coupling the control knob 1006 to the measurement member 1004. The length of the slot 1010, along the axis of the insertion member 1002, provides a range for extending or retracting the measurement member 1004 from the distal end of the insertion member 1002.

The locking grooves 1020 are formed in the surface of the insertion member 1002 adjacent and orthogonal to the slot 1010. The locking grooves can be provided in measured intervals along the length of the slot 1010. In one implementation, each locking groove 1020 is separated by substantially one-half a centimeter. Other groove separations are possible and can be either uniform or non-uniform. The control knob 1006 can be configured to engage a locking groove 1020, for example, by rotating the control knob 1006 in the direction of a locking groove 1020. In operation, for example, once the operator has extended the measurement member 1004 to the fundus, the operator can engage the nearest locking groove 1020 to lock the control knob 1006. The uterine measurement device 1000 can then be removed and the uterine dimensions later recorded based on the position of the locked control knob 1006.

Referring now to FIGS. 11A-C and 12A-C, three implementations of a tip 1101, 1102 and 1103 are shown. The distal tips 1101-1103 include atraumatic geometry configured to resist perforation of the uterine wall 1200 by reducing stress on the uterine wall 1200. The examples of atraumatic geometry that are shown in FIGS. 8A-C include a full radius tip 1101, a chamfered tip 1102 and a concave tip 1103 respectively.

As shown in FIGS. 12A-C, different atraumatic distal tip geometries produce different axial loads on the uterine wall 1200. FIG. 12A illustrates the forces on the uterine wall 1200 (shown as arrows) by a distal tip 1101 configured as a full radius tip. FIGS. 12B and 12C similarly illustrate the forces on the uterine wall 1200 by distal tips configured as a chamfered tip 1102 and a concave tip 1103 respectively. A full radius tip 1101 as shown in FIG. 12A, resists scraping the uterine wall 1200 during insertion into the uterus, but can tend to divide tissue when an axial load is applied. A chamfered tip 1102, as shown in FIG. 12B, resists scraping the uterine wall 1200 moderately well and better resists puncturing the wall 1200 relative to a full radius tip 1101. A chamfered tip 1102 tends to create less radial force (indicated by arrows) in tissue, in comparison to a full radius tip 1101 as shown in FIGS. 12A and 12B. Concave tip 1103 can significantly protect against scraping and puncturing the uterine wall 1200 and tends not to divide tissue. As shown in FIG. 12C, although the concave tip 1103 does generate some radial forces (indicated by arrows) that develop tensile hoop stress on the outer perimeter, the hoop stress produced in the central region is compressive (indicated by arrows).

In an alternative implementation, a uterine measurement device can be provided that includes a measurement member having an end cap at the distal end that can have an open position and a closed position. The end cap can be in the closed position during insertion into the uterus. Under conditions where there is a risk of the uterine measurement device perforating the uterine wall, the end cap automatically switches to the open position. The open position provides an enlarged surface area of the distal end of the measurement member of the uterine measurement device that is in contact with the uterine wall and resists perforation of the uterine tissue.

Referring to FIGS. 13A and 13B, one embodiment of an inner or measurement member 1302 of a uterine measurement device is shown. The measurement member 1302 can be incorporated into a uterine measurement device, such as the device 100 shown in FIG. 1, in which case, the measurement member 1302 would replace the inner or measurement member 104 shown in FIG. 1. The measurement member 1302 has an open and a closed position. In FIG. 13A the measurement member 1302 is in a closed position, and is configured to facilitate insertion into a uterus. In FIG. 13B the measurement member 1302 is in an open position; the end cap 1304 of the measurement member 1302 has changed geometry from having a relatively small distal tip to having an enlarged surface area.

In the embodiment depicted, the measurement member 1302 includes an elongate member 1306 having distal and proximal ends. The elongate member 1306 is generally rigid axially yet flexible and/or malleable non-axially. As such, the elongate member 1306 is rigid in the compressive direction with respect to the elongate member's distal and proximal ends, and flexible out of a longitudinal axis of the elongate member 1306. The elongate member 1306 can be rigid in the compressive direction such that an operator is provided a tactile sensation when the fundus of the uterus is engaged.

As shown in FIGS. 13A and 13B, the end cap 1304 is connected to the distal end of the elongate member 1306. The end cap 1304 can be configured in a closed position for when the elongate member 1306 is inserted into the uterus and when sounding the uterus under normal conditions (see FIG. 13A). Additionally, the end cap 1304 is in the closed position when partially or wholly within the outer sheath (e.g., insertion member 102 in FIG. 1) of the uterine measurement device 1300. The end cap 1304 can further be configured to automatically switch into an open position of enlarged surface area when a force is applied to a distal tip 1308 of the end cap 1304 by the uterine tissue in excess of a threshold force (see FIG. 13B). That is, the surface area of the end cap 1304 projected onto a plane substantially perpendicular to a longitudinal axis of the elongate member 1306 is enlarged in the open position. In the open position the enlarged geometry of the end cap 1304 resists penetration of the uterus by the measurement member 1302. The measurement member 1302 can also include a handle 1310 connected to the proximal end of the elongate member 1306. The handle 1310 can replace or be integrated with the handle 118 coupled to the insertion member 102 of the uterine measurement device 100 shown in FIG. 1.

Referring also to FIG. 14, in the embodiment depicted, the elongate member 1306 includes a shaft 1312 and a rod 1314 disposed within the shaft 1312. The rod 1314 spans the length of the elongate member 1306 and is attached to the distal end of the end cap 1304. Referring to FIG. 15, in one embodiment the rod 1314 is attached to the distal tip 1308 of the end cap 1304 by a snap fit 1500 connection. The snap fit 1500 can be in the form of a clevis-type coupling (see FIG. 15) a threaded feature, a pin, a bonding agent or any other suitable means. Where the snap fit 1500 is a clevis snap fit, a rotational degree of freedom can be provided between the rod 1314 and the distal tip 1308 of the end cap 1304.

Referring to FIG. 16, a cross-sectional view of the handle 1310 is shown. The rod 1314 can include a hardstop 1602 attached to the rod 1314 for limiting translational movement of the rod within the handle 1310. Also shown in FIG. 16, a retainer 1604 can be attached to the rod 1314 within the handle 1310, which is described further below.

Referring to FIGS. 14 and 15, the end cap 1304 can include one or more deployable fins 1400 that provide a convertible arrangement for the end cap 1304 between a closed position (see FIG. 14) and an open position (see FIG. 15). The open position provides an enlarged surface area at the distal end of the measurement member 1302. Deployment of the end cap 1304 to the open position is triggered when a force exceeding a threshold force is exerted on the distal tip 1308 of the end cap 1304 and transmitted down the shaft 1312. That is, when the measurement member 1302 reaches the end of the uterus, or another portion of uterine wall, and an operator continues pushing on the proximal end of the measurement member 1302, if the resisting force exerted by the uterine wall on the end cap 1304 exceeds the threshold force, then the open position is triggered.

As shown in FIG. 15, in one embodiment, when the open position is triggered, two fins 1400 deploy radially outwardly to provide an enlarged surface area. The fins 1400 can be formed from shorter links 1410 and longer links 1412. The length of the shorter links 1410 relative to the longer links 1412 can follow an approximate 1:3 ratio. Additionally, where the deployed shorter links 1410 are substantially perpendicular to the long axis of the measurement member 1302, the longer links 1412 are disposed at an angle including but not limited to, for example 25-30 degrees. In one embodiment, the shorter links 1410 are approximately 0.25 to 1 centimeter in length, while the longer links 1412 are approximately 0.75 to 3 centimeters in length. In another embodiment, the shorter links 1410 are approximately 0.7 centimeters in length and the longer links 1412 are approximately 2.1 centimeters in length. The outward deployment of the shorter links 1410 can include rotation of the shorter links 1410 through a larger angle than that rotated through by the connected longer links 1412. Particularly, the shorter links 1410 can be configured to deploy substantially 90 degrees to the long axis of the elongate member 1306, while the longer links 1412 deploy substantially 30 degrees to the long axis of the elongate member 1306 (see FIG. 15). The deployed shorter links 1410 and longer links 1412 create a substantially rigid, stable triangular configuration capable of withstanding substantial loads without buckling.

The shorter links 1410 and longer links 1412 of the fins 1400 can be injection molded links, pinned rigid links, resilient wire or other suitable formed links. When the end cap fins, 1400 are injection molded, the end cap 1304 can have one or more slots 1416 defining fin 1400 width and one or more holes 1414 in the slot 1416. The holes 1414 are configured to define shorter link 1410 and longer link 1412 length, and provide an area of increased bending stress, thereby providing a “living hinge” at the ends of the fins 1400. A living hinge can be, for example, a molded thin flexible bridge of material (e.g., polypropylene or polyethylene) that joins two substantially rigid bodies together. Additionally, one or more holes 1418 in the end cap 1304 located adjacent to the one or more slots 1416, can be configured to enhance the living hinge separating the shorter links 1410 and longer links 1412.

The measurement member 1302 includes a feature to sense when to switch from a closed to an open position, and a feature to deploy into the open position. In the embodiment shown, a mechanical deployment mechanism both senses when a threshold force is exceeded and automatically deploys the fins 1400 into the open position. Referring again to FIGS. 13 and 16, the deployment mechanism can be a mechanical assembly, housed within the handle 1310. The handle 1310 is attached to the elongate member 1306 at or substantially near to the proximal end. Other deployment mechanisms for converting from the closed position to the open position can be used, including electrical means by incorporating a force sensitive resistor (FSR) at the distal tip 1308. When the force exerted against the FSR exceeds a threshold value, the resistance of the FSR changes from one state to a different state. A detector located, for instance, in the handle 1310 can detect the change and trigger the release of a braking means holding the rod 1314 in place, allowing the end cap 1304 to deploy. Still another embodiment could employ a pneumatic means, whereby the force applied at the distal tip translates through the rod 1314, which could in turn bear on a plunger in a reservoir inside handle 1310. When the pressure inside the reservoir reaches the threshold value, a pressure releasing means could trigger the end cap 102 to change to its deployed condition.

An orientation indicator can be provided to indicate to an operator the proper orientation of the measurement member 1302 relative to the uterus. For example, where the fins 1400 of the measurement member 1302 deploy in a plane, the proper orientation substantially aligns the plane with the plane of the substantially flat uterus to ensure safe deployment of the fins 1400. The orientation indicator can be positioned substantially near the proximal end of the measurement member 1302. The orientation indicator can be a marking on the surface, or a tactile indicator at the proximal end of the measurement member 1302. In one embodiment, the proximal end of the handle 1310 can include an orientation indicator in the form of a flattened planar side that coincides with the plane of deployment of the fins 1400. In one embodiment, the plane of handle 1310 itself can indicate the plane of deployment of the fins 1400. Additionally, the orientation indicator can be positioned on the outer sheath of the uterine measurement device 1300 (e.g., insertion member 102 of FIG. 1) or on the control knob (e.g., control knob 106 of FIG. 1).

In the embodiment shown in FIG. 16, the mechanical assembly included within the handle 1310 includes journals 1606 for providing a single translational degree of freedom to the rod 1314, and a boss 1608 for contacting the hardstop 1602 of the rod 1314, thereby limiting the translational movement of the rod 1314. The mechanical assembly further includes a means to govern the threshold force required to trigger conversion to the open position, e.g., to deploy the fins 1400. In the embodiment depicted, the means for governing the threshold force include a spring 1610, e.g., a compression spring. The spring 1610 can be preloaded between the handle wall 1612a at the handle's proximal end and the retainer 1604 connected to the rod 1314 near the handle wall 1612b at the handle's distal end. The retainer 1604 is constrained by the adjacent handle wall 1612b to maintain the spring 1610 preload. Alternatively, the governing means can include a pressurized gas in a cylinder formed within handle 1310, wherein retainer 1604 can be configured as a piston capable of translating through the cylinder.

When a uterine measurement device incorporating a measurement member 1302 as shown in FIGS. 13A-B is inserted into a uterus, and the distal tip 1308 of the end cap 1304 presses against a uterine wall, a resistance force exerted by the uterine wall 1200 (see FIG. 12A-C) on the distal tip 1308 is transmitted along the rod 1314 to the retainer 1604. Typically, measurement of the uterus dimensions presents little risk of perforation using the uterine measurement device, since the end of the uterus can be identified by tactile sensation without exceeding the threshold force.

Under certain circumstances, e.g., through inadvertence, accident, anatomical divergence or stenosis of the uterus, the measuring process can result in forces on the uterine wall 1200 that could perforate the uterus with the uterine measurement device. Once a force approaching, but substantially lower than a force capable of perforating the uterine wall 1200, i.e., the threshold force, is transmitted to the retainer 1604, the force preloaded in the spring 1610, i.e., the threshold force, begins to compress the spring 1610. As the spring 1610 compresses, the retainer 1604 moves away from the adjacent handle wall 1612b and translates the rod 1314 through the journals 1606. The rod's translation is limited by the hardstop 1602 contacting the boss 1608. The translation of the rod 1314 relative to the shaft 1312 draws the distal tip 1308 of the end cap 1304 toward the handle 1310, thereby deploying the fins 1400 (see FIG. 15) and creating the desired enlarged surface area for resisting penetration of the end cap 1304 into the uterine wall 900.

After deployment, the fins 1400 of the measurement member 1304 can be returned to the undeployed state by e.g., physically pushing the proximal end of the rod 1314 to the undeployed position in the elongate member 1306, thereby returning the distal tip 800 of the end cap 1304 and accordingly the fins 1400 to their undeployed positions. Alternatively, in the embodiment depicted, once the force on the distal tip 1308 of the end cap 1304 is released, i.e., is less than the threshold force, the spring 1610 expands and automatically contracts the fins 1400. Once returned to the undeployed position, the uterine measurement device can safely be removed.

Referring again to FIGS. 13 and 16, the measurement member 1302 can optionally include an indicator to indicate to an operator of the uterine measurement device that the threshold force was exceeded and that the measurement member 1302 has converted to the open position. In the embodiment depicted, the indicator is a protrusion 1614 from the handle 1310 that is continuously connected to the rod 1314. When the threshold force of the measurement member 1302 is exceeded, translation of the rod 1314 causes the protrusion 1614 to protrude further from the handle 1310, thereby providing a signal or alert to the operator. In other embodiments, the indicator can be both visual and audible and can be a mechanical or an electric device or a combination of the two. For example, where the indicator is the protrusion 1614, a colored section (e.g., yellow or red) can be revealed upon exceeding the threshold force when the indicator is caused to protrude further from the handle 1310 (not shown).

Alternative techniques can also be used to provide the measurement of the uterine cavity dimensions. For example, electronic circuitry can be used. In one implementation, electrical contacts can be positioned at a predefined spacing along the outer sheath. The spacing interval can correspond to a desired measurement interval. Additionally, the interval can decrease as the distance from the proximal end of the outer sheath increases in order to provide increased measurement accuracy within a typical uterine cavity length and/or depth range. Corresponding electrical contacts can be positioned on an interior surface of the control knob (e.g., positioned on the surface of the inner circumference of a ring shaped control knob). As the control knob moves along the outer sheath, the electrical contacts of the control knob mate with corresponding electrical contacts of the outer sheath in order to complete an electrical circuit. Logic associated with the various circuit pathways can determine the distance traveled along the outer sheath by the control knob according to which electrical contacts on the outer sheath were activated. The distance the control knob advanced is used to determine the uterine cavity length. In one implementation, a display, e.g., an LCD screen, can be used to provide a digital display to an operator of the uterine cavity length.

In another implementation, the control knob can include an array of micro-switches positioned on the inner surface. Each micro switch can be configured to be switched on or off depending on whether the switch is in a raised or lowered position. The outer shaft can include an array of dimples along the outer shaft at predefined intervals. The interval can correspond to one or more desired measurement intervals. One or more of the micro switches can be toggled into the raised or lowered position at each measuring interval. In one implementation, the pattern of raised or lowered micro switches at a given measurement interval corresponds to a particular uterine length value. For example, for an array of 10 micro switches along the interior circumference of the control knob, at the first measuring interval (e.g., 1 cm), the outer shaft can have only one dimple such that only a single micro-switch is toggled, providing a signal corresponding to a length of 1 cm. At the next measuring interval (e.g., 1.5 cm), the outer sheath can have two dimples such that two micro-switches are toggled. Subsequent dimple patterns correspond to subsequent length measurement. In one implementation, the dimples at each interval are elongated to span between to the next measurement interval. The above examples are exemplary only; other electronic devices can be used to measure and/or display the uterine cavity length.

Additional embodiments disclosed herein include uterine sounding devices and methods for measurement that reduce the need for tactile feedback through use of internal visualization devices. For example, known hysteroscopes carry optical and light channels or fibers for visualization of internal tissue, structures, etc. In some embodiments, an insertion member is constructed and arranged to guide a hysteroscope within a channel having measurement graduations. Graduations provided on the insertion member can be visualized by the hysteroscope in conjunction with internal tissue boundaries including, for example, the internal cervical os and the external cervical os. Using the graduations provided on the insertion member and the visualized tissue boundaries, a physician can measure the length of the uterine cavity, depth of the uterine cavity, and/or measure the length of the endocervical canal.

FIG. 17 illustrates an example embodiment of an insertion member 1702 for a uterine sounding device (not illustrated). The insertion member 1702 includes a lumen 1730 having a proximal end 1725 and distal end 1727. The lumen can include a hollow channel 1732 that extends from the proximal end 1725 to the distal end 1727 of the insertion member 1702. The insertion member 1702 is constructed and arranged to facilitate insertion of the insertion member into and through the endocervical canal.

According to one embodiment, the insertion member 1702 can include a distal tip 1716 at the distal end of the elongated lumen 1703. In one example, the insertion member includes a set of graduations 1714 that can be used as measurement references in any measurement. For example, the set of graduations 1714 can be configured to provide a measurement of a visualized endocervical canal. In some embodiments, the set of graduations 1714 can extend from the distal tip 1716 of the insertion member 1702 along the lumen 1730 enabling measurement of the length of the uterine cavity. According to aspects and embodiments, the insertion member 1702 is constructed and arranged of a translucent or optically transparent material.

In one implementation of the insertion member 1702, a physician can advance the distal tip 1716 of the insertion member 1702 through the cervical canal and into the uterine cavity until the distal tip contacts the fundus. With the distal tip 1716 of the insertion member in place at approximately the fundus, the physician can operate a hysteroscope 1730 within the hollow channel 1732 of the insertion member to determine, for example, the boundaries of the cervical canal and the uterine cavity and the length of any of the cervical canal and the length of the uterine cavity by ascertaining the boundaries of the cervical canal and uterine cavity and the markings 1714 on the insertion member. The markings 1714 on the insertion member can be positioned on the interior of the channel 1732 to assist in visualization. Further, the distal tip 1716 can be constructed of a color material to assist in visualization of tissue boundaries and/or the positioning of the insertion member 1702. In some embodiments, markings can be provided on the interior and exterior of the insertion member. The interior marking can be positioned to provide for measurement of one dimension of an internal boundary, and the exterior markings can be positioned to provide for measurement of another dimension of an internal boundary.

It is to be appreciated that according to certain aspects and embodiments, the hysteroscope can be configured to capture images, including video, digital images, digital video images, and the like of internal tissue and/or tissue boundaries through the translucent material of the insertion member 1702. For example, the hysteroscope and insertion member can be used with a video recording device to capture images of the boundaries of the cervical canal and the length of any of the cervical canal and the depth of the uterine cavity by capturing images of the boundaries of the cervical canal and the uterine cavity and also capturing the markings 1714 on the insertion member as measurement references in any captured image. Thus, this arrangement, of the hysteroscope and insertion member can be configured to enable measurement uterine cavity length and sounding length. In some cases, both measurements can be obtained, for example, by capturing an image and proximal graduation from the set of graduations 1714 relative to the internal and external cervical os. In other embodiments, multiple images of each boundary can be captured enabling visual generation of length by a physician reviewing the captured images.

According to various aspects and embodiments, the distal tip 1716 can be an atraumatic tip, as discussed above. In some embodiments, the distal tip 1716 can also be constructed and arranged of a translucent and/or optically transparent material. In yet other embodiments, the distal tip can be opaque or colored to facilitate visualization of the tip during measurements. In further embodiments, the proximal end of the insertion member can be attached to a handle (not shown) for better gripping. Additionally, the structures discussed above with respect to deploying atraumatic tips can be disposed within such a handle.

Insertion member 1702 can also include portions where lumen 1703 has a concave section 1719. The concave section can be configured to facilitate use of the hysteroscope 1730. For example, the concave section can facilitate viewing of tissue and tissue boundaries by providing an area where the hysteroscope can visualize tissue directly (i.e., not through the transparent material of the insertion member 1702).

In FIG. 18, there is illustrated an embodiment of a uterine measurement device 1800 having an insertion member 1802, a lumen 1803 that defines an open channel 1805 between a proximal end 1825 and a distal end 1827 of the insertion member 1802. Shown at line A is a cross section of channel 1805 when viewed from the distal end. Shown in dashed line of view A is a cross section view of a hysteroscope 1830 configured to mate with the insertion member at channel 1805.

In some implementations, the insertion member 1802 can be configured for transcervical insertion into a patient. For example, a physician can insert the insertion member 1802 by using handle 1818. The physician can position the distal tip 1816 of the insertion member at approximately the fundus of the patient's uterine cavity by detecting tactile feedback at the handle 1818. In one example, once the distal tip 1816 is positioned at approximately the fundus, the hysteroscope can provide images of the patient's internal tissue. For example, the hysteroscope can communicate captured images to a monitor or other display or to a computer or recordation device for storage. The insertion member 1802 can include a set of graduations 1814 that provide measurement references of any image generated by the hysteroscope, including any imaged tissue and/or tissue boundary. By capturing an image of, for example, tissue boundaries within the patient and a proximate graduation marking, the physician can generate respective measurements for lengths of internal structures. In one example, the physician can measure an endocervical canal length and/or a uterine cavity length using imaged graduations proximate to the internal cervical os and the external cervical os.

According to aspects and embodiments, the insertion members (e.g., 1702 and 1802) disclosed herein can be configured to couple to a hysteroscope already positioned within a patient's cervix, endocervical canal, and/or uterine cavity. For example, the hysteroscope and insertion member 1802 can be inserted transcervically with the hysteroscope resting within a channel of the insertion member (e.g., 1805). As discussed, the hysteroscope can be used to visualize graduations on the insertion member 1802 to obtain measurements of internal tissue boundaries.

FIG. 19 illustrates an embodiment of a uterine measurement device 1900 having an insertion member 1902 and a transparent lumen 1903 that defines a channel 1905 between a proximal end 1925 and a distal end 1927 of the insertion member 1902. The measurement device is configured to mate with a hysteroscope by positioning channel 1905 over the hysteroscope. In one example, the channel 1905 is positioned over a hysteroscope that is being used to visualize internal tissue of a patient. The uterine measurement device 1900 includes graduations at 1914 that can be captured by the hysteroscope to obtain measurements of, for example, internal tissue boundaries. The uterine measurement device can be positioned to capture an endocervical canal length and/or a uterine cavity length among other examples. In some alternative approaches, the uterine measurement device 1900 can be inserted into the patient and the hysteroscope can be introduced through the channel 1905.

As has been described herein, various embodiments of a uterine measurement device (including e.g., 1800, and 1900, and FIG. 17) can be used in conjunction with a hysteroscope to enable visualization of internal distances by references to graduations on the measurement devices. FIG. 20 illustrates an embodiment of process 2000 for obtaining a measurement of internal distance using a hysteroscope and any of the uterine measurement devices disclosed herein. In process 2000, a measurement device is used to measure, for example, the length of a uterine cavity (endometrial cavity) and/or a uterine sounding length based on hysteroscopic procedures.

In some embodiments, the measurement device can be as simple as a one-piece device with an atraumatic tip (discussed above) for placement against the fundus of the uterus. An optically transparent or translucent shaft can be used to enable concurrent use with a separate hysteroscope. The optically transparent or translucent shaft can be constructed with an open channel (e.g., semi-circular shaft) or hollow tube in which the hysteroscope can travel. The hysteroscope can then be used to visualize the transition from endometrium to cervix, and graduations engraved or marked on the shaft can be configured to indicate the distance from the transition to, for example, the fundus. In some embodiments, the graduations may extend so that the uterine sounding dimension (e.g., length from the fundus to the external cervical os) may also be determined with the hysteroscope by a visualization of internal tissue boundaries and proximate graduations. In various embodiments, a handle may be included for assistance with placement and manipulation of the measurement device.

Process 2000 begins at 2001 with positioning the measurement device on the hysteroscope, such that the hysteroscope is mated to the measurement device within a hollow or opening. At 2002 the hysteroscope and measurement member are inserted transcervically. The measurement device is advanced into the patient at 2004 until the measurement device has passed at least into the uterine cavity. The hysteroscope can be used to capture images of graduations proximate to internal tissue boundaries during insertion and once the measurement device is in position. For example, at 2006, the hysteroscope can capture one or more images of the patient's external cervical os and internal cervical os. The one or more images can include a capture of graduations on the measurement device proximate to either boundary. The relative spacing between the graduations on the measurement device can be used to ascertain a measure of, for example, an endocervical length at 2008. Optionally, the measurement device can be extended at 2004 until a distal tip of the device (e.g. 1716 and 1816) contacts the fundus of the patient. With the distal tip positioned at the fundus, the capture of images of the graduations on the measurement device at 2006 enables measurement of the length of uterine cavity (e.g., distance between the fundus and the internal and/or external cervical os) as well as measurement of the length of the endocervical canal at 2008.

One should appreciate that process 2000 can include additional steps or be executed in different order. In some embodiments, process 2000 can include acts of placing a hysteroscope in position and then inserting the measurement device transcervically. In some implementations, the measurement device is configured to be positioned around the body of the hysteroscope and then advanced into the patient. In one implementation, a physician can perform hysteroscopic procedures, and then introduce the measurement device for imaging of tissue boundaries and proximate graduations.

It is to be appreciated that embodiments of the methods and apparatuses discussed herein are not limited in application to the details of construction and the arrangement of components set forth in the following description or illustrated in the accompanying drawings. The methods and apparatuses are capable of implementation in other embodiments and of being practiced or of being carried out in various ways. Examples of specific implementations are provided herein for illustrative purposes only and are not intended to be limiting. In particular, acts, elements and features discussed in connection with any one or more embodiments are not intended to be excluded from a similar role in any other embodiments.

Also, the phraseology and terminology used herein is for the purpose of description and should not be regarded as limiting. Any references to embodiments or elements or acts of the systems and methods herein referred to in the singular may also embrace embodiments including a plurality of these elements, and any references in plural to any embodiment or element or act herein may also embrace embodiments including only a single element. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. Any references to front and back, left and right, top and bottom, upper and lower, and vertical and horizontal are intended for convenience of description, not to limit the present systems and methods or their components to any one positional or spatial orientation.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A uterine measurement device, comprising:
an insertion member having a distal end, a proximal end, and a lumen, the distal end of the insertion member being configured for insertion into and through an endocervical canal and into an opening of a uterine cavity;
an expansion member disposed near the distal end of the insertion member, the expansion member being constructed and arranged to selectively have an expanded state that allows for insertion of the distal end of the insertion member through the endocervical canal and into the opening of the uterine cavity and configured to have an expanded state having a geometry larger than the opening to the uterine cavity; and
a measurement member moveable relative to the insertion member, wherein the measurement member is constructed and arranged to selectively extend beyond the distal end of the insertion member, wherein the measurement member is at least as long as the uterine cavity.

2. The uterine measurement device of claim 1, wherein the expansion member has a diameter of about 0.575 inches in the expanded state.

3. The uterine measurement device of claim 1, wherein the expansion member has a diameter of greater than about 0.454 inches to less than about 0.680 inches in the expanded state.

4. The uterine measurement device of claim 1, wherein the expansion member has a diameter of greater than about 0.533 inches and less than about 0.589 inches in the expanded state

5. The uterine measurement device of claim 1, wherein the expansion member includes deployable wings.

6. The uterine measurement device of claim 5, wherein the deployable wings in the deployed state are configured to collapse in response to exceeding a threshold seating force.

7. The uterine measurement device of claim 5, further comprising a deployment mechanism configured to selectively transition the deployable wings between a contracted state and a deployed state.

8. The uterine measurement device of claim 7, wherein the deployment mechanism includes at least one of a ring structure configured to deploy the deployable wings in response to rotation, a spring, and a collar configured to accept axial force and redirect the axial force into lateral force upon the deployable wings.

9. The uterine measurement device of claim 1, further comprising a tether connected to a distal end of the expansion member, wherein the expansion member is configured to transition between a contracted position and an expanded state responsive to application and release of force directed through the tether.

10. The uterine measurement device of claim 8, wherein the expansion member is configured to seat near the internal os at a location within about 0.5 cm by applying a target seating force.

11. The uterine measurement device of claim 9, wherein the seating force is about 0.4 to 2.0 lbs.

12. The uterine measurement device of claim 1, wherein the insertion member further comprises graduations marked on at least a portion of a length of the insertion member for indicating relative movement of the measurement member to the insertion member, the relative movement corresponding to the measurement of the depth of the uterine cavity.

13. The uterine measurement device of claim 12, further comprising:
a control knob located near the proximal end of the insertion member; and
a slot formed in a proximal region of the insertion member and adjacent to the graduations and configured to receive the control knob, wherein the control knob is movable within the slot to advance and retract the measurement member within the lumen of the insertion member, wherein a position of the control knob relative to the graduations indicates the dimensions of the uterine cavity with the distal end of the measurement member positioned at approximately the fundus of the uterus.

14. A method of using a uterine measuring device, the uterine measuring device including an insertion member and a measurement member, the measurement member slideably disposed within the insertion member, the insertion member including an expansion member, the method comprising:
advancing the uterine device transcervically until a distal end of the insertion member is within a uterine cavity;
expanding the expansion member within the uterine cavity into an expanded state;
moving the expansion member until the expansion member contacts an internal cervical os without passing proximally through the internal os;
moving the measurement member until the measurement member contacts a fundus; and
determining a dimension of the uterine cavity based on a relative position of the insertion member and the measurement member.

15. The method of claim 14, wherein the act of contacting the internal cervical os without passing proximally through the internal os includes applying a seating force of less than about 2.0 lbs.

16. The method of claim 14, wherein the act of moving the expansion member includes contacting the internal cervical os with a seating force greater than about 0.4 lbs.

17. The method of claim 14, further comprising withdrawing the uterine measuring device from the uterine cavity with the expansion member in an expanded state by applying a seating force of at least about 4.0 lbs.

18. The method of claim 14, wherein the act of expanding the expansion member includes an act of actuating a tether connected to the expansion member, and wherein the method further comprises actuating a deployment mechanism configured to transition deployable wings between a contracted position and a deployed position.

19. (canceled)
